(19) United States
(12) Patent Application Publication
(10) Pub. No.: US 2010/0196342 A1
(43) Pub. Date: Aug. 5, 2010

(54) MOOD, MEMORY AND COGNITIVE FUNCTION WITH PYRIDOXAL 5'-PHOSPHATE

(76) Inventors: Albert Friesen, Winnipeg (CA); Dawson James Reimer, Winnipeg (CA)

Correspondence Address: MERCHANT & GOULD PC, P.O. BOX 2903, MINNEAPOLIS, MN, 55402-0903, US

(21) Appl. No.: 12/602,435

(22) PCT Filed: May 28, 2008
(86) PCT No.: PCT/CA2008/001025

§ 371 Date: Apr. 13, 2010

(30) Foreign Application Priority Data
May 28, 2007 (CA) 2,590,605

(51) Int. Cl. A61K 31/675; A61P 25/24; A61P 25/28

(52) U.S. Cl. 424/94.1

(57) ABSTRACT

This invention relates to improvement of mood, memory and cognitive function with administration of pyridoxal 5′-phosphate. Also provided is the use of pyridoxal 5′-phosphate for the preparation of a medicament for the improvement of mood, memory and cognitive function, and kits comprising pyridoxal 5′-phosphate for the same purpose.

### CROSS-REFERENCE TO RELATED APPLICATION

This application claims priority to and the benefit of Canadian Patent reference.

### FIELD OF THE INVENTION

This invention relates to improvement of mood, memory and cognitive function with administration of pyridoxal 5′-phosphate.

### BACKGROUND OF INVENTION

Long term memory function, in terms of memory storage and retrieval as well as memory processing speed, is observed to decline with age. Long term memory is believed to be the most vulnerable component of memory affected by the natural aging process.

The elderly are commonly found to have vitamin deficiencies since they consume smaller quantities of food as compared to younger individuals and so their nutritional needs are not met. It is well known that vitamin deficiencies may lead to decreased mental function and may be causal regarding age-related cognitive impairment (Malouf and Grimley Evans, 2003). It has been suggested that a long term benefit may be observed due to administration of a vitamin B6 supplement thereby decreasing the development of dementia in the elderly (Malouf and Grimley Evans, 2003).

A study among males, ages 70-79 years, receiving vitamin B6 supplementation at 20 mg daily for 3 months revealed improved information storage as compared to the age, plasma P5P level and intelligence score matched placebo group.

An associative learning task revealed improved long term memory storage based on the ability of the subjects to remember the information presented as opposed to being ‘forgetful.’

Vitamin B6 has also been shown to improve mood and combat depression (Malouf and Grimley Evans, 2003).

Pyridoxine is absorbed from the digestive tract where it is transported to the liver and oxidized to form pyridoxal. It is then phosphorylated by pyridoxal kinase to form pyridoxal 5′-phosphate (P5P) (Vermaak et al., 1988). Administration of pyridoxine to treat conditions of the previously mentioned disorders has shown to improve healing, however the effects may be limited based on the dose.

Pyridoxine has been known to produce toxicity at high doses, and limits plasma P5P levels and and the U.S. National Academy of Sciences has therefore recommended 100 mg as the Tolerable Upper Intake Level (unlikely to pose risks of adverse health effects in healthy people) for pyridoxine (National Academy of Science, 1998). With respect to safety, the main toxicity associated with vitamin B₆ is neurotoxicity. The administration of pyridoxine results in high plasma levels of pyridoxine which can lead to toxic effects such as peripheral neuropathy (Schaumburg et al, 1983). The sensory neuropathy that occurs following administration of large dose levels of oral pyridoxine has been attributed to high circulating concentrations of pyridoxine per se. High concentrations of pyridoxine lead to the inhibition of P5P binding to apoenzymes resulting in decreased levels of plasma P5P (the active form) (Bassler, 1988) and thus greater levels of P5P are achieved by its direct administration.

Previous investigators state that a higher dosage of vitamin B6 (300 mg) would produce results with even greater significance and that this improvement will continue as the dose of pyridoxine increases (Deijen et al., 1992). However, there is significant hesitancy in administering such high dosages of vitamin B6, due to its toxic nature and adverse side effects.

It is believed that this role for vitamin B6 is through its metabolite, pyridoxal 5′-phosphate, which is thought to be involved with adequate memory function due to its role in the biosynthesis of a variety of neurotransmitters.

Typically, with most drugs, increasing the dosage of the drug is desirable, rendering the drug more efficacious. This increase in dosage is mitigated by side effects and toxicity, which typically increase with dosage.

Pyridoxal-5′-phosphate (3-hydroxy-2-methyl-5-[(phosphonooxy)methyl]-4-pyridine-carboxaldehyde, or “P5P”) is a naturally occurring metabolite of pyridoxine and is formed in mammalian cells by phosphorylation and oxidation reactions. A recent evaluation demonstrated that P5P inhibits adenosine triphosphate (ATP) induced calcium ion influx into cells. Results suggest that this action is due to an inhibition of purinergic receptors known as P2 purinoceptors.

P5P can be chemically synthesized in a number of ways, for example, by the action of ATP on pyridoxal, by the action of phosphorus oxychloride on pyridoxal in aqueous solution, and by phosphorylation of pyridoxamine with concentrated phosphoric acid followed by oxidation.

It would be desirable to have a treatment resulting in improvement of mood, memory and cognitive function, for example, a treatment for depression, dementia, or for improved long term memory storage in aged patients, that can be given at relatively high concentrations, with a minimum of side effects or toxicity.

### SUMMARY OF THE INVENTION

In one embodiment of the present invention is provided a method for the treatment of depression in a mildly depressed mammal comprising administration of pyridoxal 5′-phosphate.

In another embodiment of the present invention is provided a method for the improvement of long term memory storage in elderly mammals, comprising administration of pyridoxal 5′-phosphate.

In yet another embodiment of the present invention is provided a method for the prevention of or for the slowing of the progression of dementia in a mammal comprising administration of pyridoxal 5′-phosphate.

In one aspect, the mammal is a human and the administration is an oral administration of between 100-4000 mg/day, for example, between 100-750 mg/day or about 250 mg/day.

In another embodiment of the present invention is provided the use of P5P in the preparation of a medicament for the treatment of depression.

In yet another embodiment of the present invention is provided the use of P5P in the preparation of a medicament for the improvement of long term memory storage.

In yet another embodiment of the present invention is provided the use of P5P in the preparation of a medicament for the prevention of or the slowing of the progression of dementia.

In one aspect, the medicament comprises between 100-4000 mg of P5P, for example, 100-750 mg of P5P or about 250 mg of P5P.

In another embodiment of the present invention is provided the use of P5P for the treatment of depression.

In yet another embodiment of the present invention is provided the use of P5P for the improvement of long term memory storage.

In yet another embodiment of the present invention is provided the use of P5P for the prevention of or the slowing of the progression of dementia.

In one aspect, the use comprises administration of between 100-4000 mg per day of P5P, for example, 100-750 mg/day or about 250 mg/day.

In another embodiment of the present invention is provided a kit comprising: (a) a pharmaceutical preparation for oral administration comprising pyridoxal 5′-phosphate; (b) instructions for the administration of said preparation; wherein said instructions specify that the preparation is to be administered daily to an elderly patient for the prevention of or the slowing of the progression of dementia.

In another embodiment of the present invention is provided a kit comprising: (a) a pharmaceutical preparation for oral administration comprising pyridoxal 5′-phosphate; (b) instructions for the administration of said preparation; wherein said instructions specify that the preparation is to be administered daily to an elderly patient for improvement of long term memory storage.

In another embodiment of the present invention is provided a kit comprising: (a) a pharmaceutical preparation for oral administration comprising pyridoxal 5′-phosphate; (b) instructions for the administration of said preparation; wherein said instructions specify that the preparation is to be administered daily for the treatment of depression.

In one aspect of the present invention, the instructions further specify that the preparation should be administered in a dosage range of between 100-4000 mg/day, for example, between 100-750 mg/day, or 250 mg/day.

### DETAILED DESCRIPTION

The present inventors have surprisingly found that pyridoxal 5′-phosphate is safe and low in side effects at relatively high concentrations, including concentrations previously known to be undesirable for vitamin B6.

The present inventors have found that P5P is thus more tolerable than vitamin B6 and has a much higher dosage requirement to result in toxicity, such as is observed with high doses of vitamin B6. High doses of P5P is recognized as an improvement in treatment.

### EXAMPLE 1

### Safety and Tolerance of Pyridoxal-5′-Phosphate Enteric-Coated Tablet

A Phase I, single center, single-dose, open-label, sequential ascending dose study to evaluate the safety and tolerance of pyridoxal-5′-phosphate, in an enteric-coated tablet, following a single dose of 250 mg, 750 mg, 1000 mg, and 4000 mg in healthy subjects under fasting conditions was conducted.

A total of 32 healthy, adult subjects signed the study-specific Informed Consent Form and were confined in the clinical study unit; of these subjects, 24 (6 subjects in each dose level; 3 males and 3 females) were dosed and were enrolled in the study; all of these enrolled subjects completed the study. Subjects were confined to the SFBC Anapharm Clinical Research Facility from at least 12 hours prior to drug administration until after the 24.0-hour post-dose blood draw.

Subjects enrolled in this study were members of the community at large. Subject screening procedures included informed consent, inclusion/exclusion check, demography, medical history, medication history, physical examination, height, weight, body mass index, a concomitant medication check, vital signs measurements (blood pressure, pulse rate, respiratory rate, and oral temperature), a 12-lead electrocardiogram (ECG), a urine drug screen, a urine pregnancy test (female subjects), hematology, biochemistry, urinalysis, and HIV and hepatitis testing. All participating subjects were judged eligible for the study when assessed against the inclusion and exclusion criteria.

All cohorts were sequentially dosed in an ascending fashion. Subsequent cohorts were dosed only after the completion of clinical part of the previous cohort and after revision, by the Sponsor and the Qualified Investigator, of the safety data.

Subjects were administered a single oral dose of study medication, as a 1×250 mg (Cohort 1), 3×250 mg (Cohort 2; total dose of 750 mg), 4×250 mg (Cohort 3; total dose of 1000 mg), or 16×250 mg (Cohort 4; total dose of 4000 mg) enteric-coated tablets.

After a supervised overnight fast of at least 10 hours, subjects were administered the medication as a single oral dose of 1, 3, 4, or 16 enteric-coated tablets containing 250 mg of P5P (total dose of 250 mg, 750 mg, 1000 mg, or 4000 mg), with 300 mL of water. Subjects were dosed as specified in the protocol, and subsequently fasted for at least 4 hours. Subjects in Cohorts 2 to 4 did not receive their dose until the clinical part of the preceding dose level was completed, the safety data reviewed by the Principal Investigator and the Sponsor, and a decision taken to proceed or not with the next dose level.

Clinical laboratory tests (hematology, biochemistry, and urinalysis) were performed for each subject at the time of the screening and post-study procedures and prior to dosing.

The pharmacokinetic parameters to determine bioavailability for this study were: area under the concentration-time curve from time zero to time of last non-zero concentration (AUC₀₋ₜ), maximum observed concentration (Cₘₐₓ), time of observed Cₘₐₓ (Tₘₐₓ) and elimination half-life (equivalent to t_{1/2}).

P5P produced optimal effects when administered at 250 mg likely due to a directly proportional relationship between the dose administered and the variability of plasma P5P concentrations achieved in a subject (Table 1), most likely due to unknown enzymatic activities.

TABLE 1
Summary of baseline-corrected pharmacokinetic parameters for the
enteric-coated tablet

| | Dose | | | |
|---|---|---|---|---|
| | 250 mg | 750 mg | 1000 mg | 4000 mg |
| AUC₀₋ₜ | 2.6 ± 1.3 | 12.8 ± 15.4 | 5.9 ± 7.7 | 6.9 ± 7.4 |
| (μg · h/mL) | | | | |
| Cₘₐₓ (μg/mL) | 0.3 ± 0.2 | 3.9 ± 5.4 | 1.5 ± 2.3 | 1.7 ± 2.7 |
| Tₘₐₓ (h) | 3.2 ± 1.3 | 2.8 ± 0.9 | 2.9 ± 1.4 | 4.7 ± 3.5 |
| T_{1/2} (h) | 53.8 ± 41.9 | 27.9 ± 19.5 | 36.2 ± 30.6 | 16.5 ± 16.5 |

All of the patients proceeded to the highest dosage form, and none of the patients presented any significant side effects or evidence of toxicity. Thus P5P was found to be well tolerated in patients in dosages up to 4000 mg.

### EXAMPLE 2

### Improvement of Information Storage with Administration of P5P

Human males aged between 65 and 90 are separated into four random groups: control, low dose of P5P (100 mg/day), medium dose of P5P (250 mg/day), and high dose of P5P (750 mg/day). All dosages are administered orally for 3 weeks before subjecting the patients to an associative learning task.

The patients are subjected to an associative learning task, as follows. Briefly, patients are comfortably seated in front of a computer. Patients are asked to learn the associations between six colors and the numbers 1-6, using trial and error. Subjects are then required to recall this association, a defined period of time after the associations are learned. Tests are repeated, with the defined period of time increasing, until the patients are no longer able to associate the colors with the numbers.

Patients receiving P5P are able to recall the association between the numbers and the colors a significantly longer time after the association is made, as compared to control patients. Further, though there is no significant difference in associative learning and memory between the medium and high dose groups, there is significant difference between the low group and these two groups.

Thus the low dose group exhibits improved information storage and retrieval than the control group, and the medium and high dose groups exhibits improved information storage and retrieval over the low dose group.

### EXAMPLE 3

### Treatment of Depression Using P5P

Individuals diagnosed with mild depression are divided into four groups at random: control, low dose of P5P (100 mg/day), medium dose of P5P (250 mg/day), and high dose of P5P (750 mg/day). All dosages are administered orally for 3 weeks.

The level of depression of the individuals is assessed, both before, and after treatment, using conventional methodologies. These include completion of several depression inventory questionnaire scales including the Cornell Scale for Depression in Dementia, Centre for Epidemiologic Studies Depression Scale, The Geriatric Depression Scale (Short Version), Beck Depression Inventory and the Zung Self-Rating Depression Scale as well as diagnosis by a trained professional according to the DSM-IV. These questionnaires measure depression through questions related to appetite, sadness, worthlessness/inadequacy, concentration, exhaustion, failure, retardation, socialization, isolation, anxiety, irritability, agitation, loss of interest, lack of energy, diurnal mood variation, insomnia, suicidal thoughts, low self-esteem, pessimism, mood congruent delusions, satisfaction, withdrawal, fulfillment, mood, fear, guilt, self-accusations, somatic preoccupation and loss of libido.

Patients given P5P are significantly less depressed than control individuals. Patients in the medium and high dose groups are significantly less depressed than patients in the low dose group. Patients in the medium and high dose groups are not significantly different.

1-24. (canceled)

25. A method for the treatment of depression in a mildly depressed mammal comprising administering pyridoxal 5′-phosphate.

26. A method for the improvement of long term memory storage in elderly mammals, comprising administering pyridoxal 5′-phosphate.

27. A method for the prevention of or for the slowing of the progression of dementia in a mammal comprising administering pyridoxal 5′-phosphate.

28. The method of claim 25 wherein the mammal is a human and the pyridoxal 5′-phosphate is between 100-4000 mg/day.

29. The method of claim 28 wherein the pyridoxal 5′-phosphate is between 100-750 mg/day.

30. The method of claim 28 wherein the administration is about 250 mg/day.

31. A kit comprising:
(a) a pharmaceutical preparation for oral administration comprising pyridoxal 5′-phosphate;
(b) instructions for the administration of said preparation;
wherein said instructions are selected from the group consisting of:
specifying that the preparation is to be administered daily to an elderly patient for the prevention of or the slowing of the progression of dementia;
specifying that the preparation is to be administered daily to an elderly patient for improvement of long term memory storage; and
specifying that the preparation is to be administered daily for the treatment of depression.

32. The kit of claim 31 wherein the instructions further specify that the preparation should be administered in a dosage range between 100-4000 mg/day.
